(19) United States
(12) Patent Application Publication
(10) Pub. No.: US 2022/0087536 A1
(43) Pub. Date: Mar. 24, 2022

(54) TEMPERATURE MEASUREMENT METHOD AND MEASUREMENT DEVICE

(71) Applicant: RADIANT INNOVATION INC., Hsinchu County (TW)

(72) Inventors: YU-CHIEN HUANG, HSINCHU CITY (TW); CHIEN-CHANG LIAO, HSINCHU CITY (TW)

(21) Appl. No.: 17/027,675

(22) Filed: Sep. 21, 2020

(51) Int. Cl. A61B 5/01; G01J 5/00; G01J 5/10; G01J 5/02; A61B 5/00

(52) U.S. Cl. CPC A61B 5/015; G01J 5/0025; G01J 5/10; G01J 5/0265; G01J 5/026; A61B 2560/0431; A61B 5/0075; G01J 2005/0077; A61B 2562/0271; A61B 2576/02; A61B 5/7203

(57) ABSTRACT

A temperature measurement method and a measurement device are provided. The temperature measurement method includes capturing, by a temperature measurement device, a first sampling temperature of a first measurement position surface of a subject body and a second sampling temperature of a second measurement position surface of the subject body, the first measurement position surface and the second measurement position surface are where body surfaces of the subject have different thermal dissipation rates when in contact with the air; computing an original measured temperature according to the first sampling temperature; computing a compensated temperature, the compensated temperature including a temperature difference compensation value that is computed according to a difference between the first sampling temperature and the second sampling temperature; and correcting the original measured temperature according to the compensated temperature to obtain the measured temperature.

### FIELD OF THE DISCLOSURE

The present disclosure relates to a temperature measurement method and a measurement device, and more particularly to a contactless temperature measurement method and a contactless measurement device to detect whether a human body temperature is normal.

### BACKGROUND OF THE DISCLOSURE

When people have a fever, the body is generally inflamed or infected. In recent years, a variety of respiratory virus infections (e.g., influenza, SARS, MERS, COVID-19) cause the body temperature to become abnormally high and show symptoms of fever. Therefore, it has become an important means of protection to measure the body temperature of people before they enter public areas in order to prevent the risk of cluster infections.

In medicine, fever is usually defined as having a body temperature above 38 Celsius degrees. Different methods used for measuring temperature produce different results, and the temperature reading depends on which part of the body is being measured, such as the mouth, the ears, the forehead, the rectum, and the armpits. In the abovementioned different body temperature measurement methods, rectal temperature is the closest measurement method to the core body temperature, however, the rectal temperature measurement is usually only suitable for infants in clinics and not suitable for temperature screening in public areas. The oral temperature and tympanic temperature are also closer to the core body temperature and have less measurement error compared to other measurement methods. However, oral temperature and tympanic temperature measurement methods require body contact with the subject and are not suitable for people in public areas due to hygienic concerns.

Currently, it is common to use a contactless temperature measurement method to measure body temperature in public areas, such as using a forehead thermometer to measure forehead temperature, or using infrared imaging equipment to screen for fever with thermal image. Contactless body temperature measurement methods can reduce the risk of infection because the measurement equipment does not contact the subject, and the body temperature measurement result can be quickly detected. However, one of the main issues of the contactless body temperature measurement method is that a surface temperature of the subject is prone to be influenced by the ambient temperature, which leads to a great amount of temperature difference between the measured body temperature and an actual core body temperature of the subject that results in a misjudgment.

In detail, the hypothalamus sets a desired body temperature and stimulates heat production and retention by skin molecular vibration, dilation, or contraction of pores and blood vessels so as to enable thermal conduction, thermal convection, thermal radiation, and water evaporation in the human body. In general, the contactless temperature measurement method is mostly used to measure facial or forehead temperature by an infrared thermometer or thermal imaging equipment, but the facial temperature is prone to be influenced by the ambient temperature because pores and blood vessels are densely distributed under the skin of the face. For example, the facial temperature of the subject might be downwardly biased when the subject moves from an outdoor environment with cold wind and low ambient temperature to indoors. Therefore, the subject should stay indoors for a while until the facial temperature returns to the temperature that is set by the hypothalamus, such that the temperature measurement error due to the ambient temperature can be reduced.

Due to the abovementioned reasons, how the temperature measurement error due to the ambient temperature can be reduced when using the contactless body temperature measurement method has become a critical topic in the industry.

### SUMMARY OF THE DISCLOSURE

In response to the above-referenced technical inadequacies, the present disclosure provides a contactless temperature measurement method and a contactless measurement device to reduce the temperature measurement error due to the ambient temperature.

In one aspect, the present disclosure provides a temperature measurement method for measuring a body temperature of a subject to obtain a measured temperature. The temperature measurement method includes capturing, by a temperature measurement device, a first sampling temperature of a first measurement position surface of the subject body and a second sampling temperature of a second measurement position surface of the subject body, the first measurement position surface and the second measurement position surface being where body surfaces of the subject have different thermal dissipation rates in the air; computing an original measured temperature according to the first sampling temperature; computing a compensated temperature, the compensated temperature including a temperature difference compensation value that is computed according to a difference between the first sampling temperature and the second sampling temperature; and correcting the original measured temperature according to the compensated temperature to obtain the measured temperature.

In certain embodiments, the first measurement position surface is located on a forehead of the subject, and the second measurement position surface is located on a cheek of the subject.

In certain embodiments, the compensated temperature further includes an equipment compensation value, the equipment compensation value being a constant value for correcting a fixed equipment error of the temperature measurement device.

In another aspect, the present disclosure provides a temperature measurement device configured to measure a body temperature of a subject to obtain a measured temperature. The temperature measurement device includes at least one temperature sensing unit, a computing unit, and a temperature compensation unit. The at least one temperature sensing unit is an infrared temperature sensor or a thermal image sensor that is configured to capture a first sampling temperature on a first measurement position surface of the subject body, a second sampling temperature on a second measurement position surface of the subject body, the first measurement position surface and the second measurement position surface being where body surfaces of the subject having different thermal dissipation rates. The computing unit is connected to the at least one temperature sensing unit, and is configured to compute an original measured temperature according to the first sampling temperature. The temperature compensation unit is configured to compute a compensated temperature for correcting the original measured temperature to obtain the measured temperature, the temperature compensation value including a temperature difference compensation value, the temperature difference compensation value being computed by a temperature difference compensation function according to a difference between the first sampling temperature and second sampling temperature.

In certain embodiments, the temperature measurement device is a handheld infrared thermometer, the temperature sensing unit having at least one infrared temperature sensor, and the first sampling temperature and the second sampling temperature being captured by the temperature sensor that is respectively moved to the first measurement position surface and the second measurement position surface of the subject body.

In certain embodiments, the temperature measurement device is a handheld infrared thermometer, the temperature sensing unit having at least two infrared temperature sensors, the first sampling temperature and the second sampling temperature being captured by the two temperature sensors that respectively aim at the first measurement position surface and the second measurement position surface of the subject body.

In certain embodiments, the temperature measurement device is an infrared thermal imager, the temperature sensing unit being a thermal image sensor or a focal plane infrared sensor configured to capture a thermal image including the subject, and the first sampling temperature and the second sampling temperature are obtained by analyzing the thermal image corresponding to surfaces of the first measurement position surface and the second measurement position surface of the subject body.

Therefore, an advantageous effect of the present disclosure is that the measurement error generated from the subject being influenced by the ambient temperature can be corrected and the contactless temperature measurement method can be conducted without waiting for the temperature of the subject to return to the temperature that is set by the hypothalamus, so that the convenience and the accuracy of the body temperature measurement operation can be improved.

These and other aspects of the present disclosure will become apparent from the following description of the embodiment taken in conjunction with the following drawings and their captions, although variations and modifications therein may be affected without departing from the spirit and scope of the novel concepts of the disclosure.

### BRIEF DESCRIPTION OF THE DRAWINGS

The present disclosure will become more fully understood from the following detailed description and accompanying drawings.

FIG. 1 is a schematic view of a temperature measurement being performed on a subject by a temperature measurement method according to an embodiment of the present disclosure.

FIG. 2 is a flowchart of the temperature measurement method according to an embodiment of the present disclosure.

FIG. 3 is a coordinate distribution of experimental data and a trend curve that is obtained according to linear regression analysis in order to compute a temperature difference compensation function according to an embodiment of the present disclosure.

FIG. 4 illustrates a coordinate distribution of experimental data and a trend curve that is obtained according to multiple regression analyses in order to compute a temperature difference compensation function according to an embodiment of the present disclosure.

FIG. 5 illustrates a system block diagram of a temperature measurement device according to an embodiment of the present disclosure.

FIG. 6 illustrates a perspective view of the temperature measurement device according to a first embodiment of the present disclosure.

FIG. 7 illustrates a schematic view of a manual operation of the temperature measurement device according to the first embodiment of the present disclosure.

FIG. 8 illustrates a perspective view of a temperature measurement device according to a second embodiment of the present disclosure.

FIG. 9 illustrates a schematic view of a manual operation of the temperature measurement device according to the second embodiment of the present disclosure.

FIG. 10 illustrates another schematic view of the manual operation of the temperature measurement device according to the second embodiment of the present disclosure.

FIG. 11A and FIG. 11B illustrates schematic views of a manual operation of measuring a back of a hand and a wrist to capture the first measured temperature and the second measured temperature using the temperature measurement method according to an embodiment of the present disclosure.

### DETAILED DESCRIPTION OF THE EXEMPLARY EMBODIMENTS

The present disclosure is more particularly described in the following examples that are intended as illustrative only since numerous modifications and variations therein will be apparent to those skilled in the art. Like numbers in the drawings indicate like components throughout the views. As used in the description herein and throughout the claims that follow, unless the context clearly dictates otherwise, the meaning of “a”, “an”, and “the” includes plural reference, and the meaning of “in” includes “in” and “on”. Titles or subtitles can be used herein for the convenience of a reader, which shall have no influence on the scope of the present disclosure.

The terms used herein generally have their ordinary meanings in the art. In the case of conflict, the present document, including any definitions given herein, will prevail. The same thing can be expressed in more than one way. Alternative language and synonyms can be used for any term(s) discussed herein, and no special significance is to be placed upon whether a term is elaborated or discussed herein. A recital of one or more synonyms does not exclude the use of other synonyms. The use of examples anywhere in this specification including examples of any terms is illustrative only, and in no way limits the scope and meaning of the present disclosure or of any exemplified term. Likewise, the present disclosure is not limited to various embodiments given herein. Numbering terms such as “first”, “second” or “third” can be used to describe various components, signals or the like, which are for distinguishing one component/signal from another one only, and are not intended to, nor should be construed to impose any substantive limitations on the components, signals or the like.

### First Embodiment

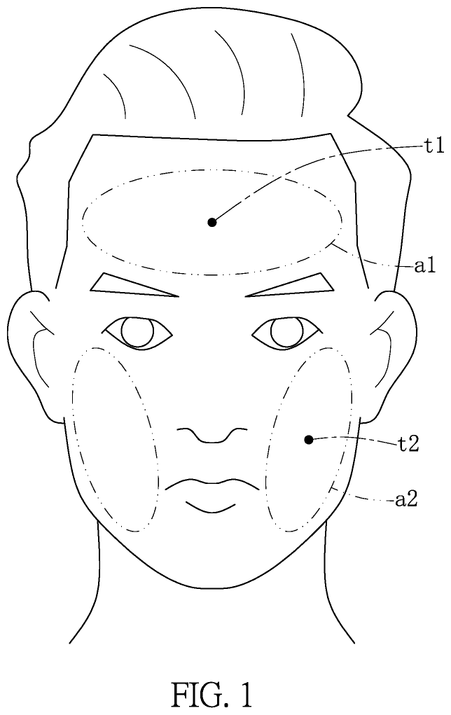

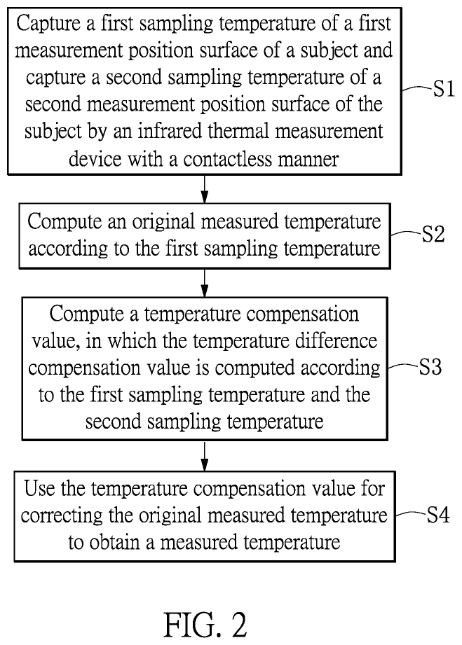

Referring to FIG. 1 and FIG. 2, the embodiment of the present disclosure provides a temperature measurement method for contactless body temperature measurement, which may be performed mainly by devices such as an infrared thermometer or infrared imaging equipment.

As shown in FIG. 1, the principle of the measurement method of the present disclosure is that a skin temperature of a human body depends on the vascular distribution and what kind of organ is under the skin, and a thermal dissipation rate varies when the skin contacts the air. For example, the skin temperature of the forehead is closest to the brain and will have the highest temperature on the face because the brain generates the most heat among the head region, and the thermal dissipation rate of the forehead temperature is low when the skin contacts the air. The skin temperature of the cheek will be lower than that of the forehead temperature because there are no human organs under the surface of the cheek, there are more blood vessels and pores in the cheek region, so the thermal dissipation rate of the cheek that is exposed to the air is higher than that of the forehead.

In other words, when the human body is in contact with cold air, the thermal dissipation rate varies with the position (e.g., forehead and cheek) of the body surface, so that a temperature difference between two positions of the body surfaces becomes greater due to different thermal dissipation rates when the body contacts the air. Therefore, the measurement method of the present disclosure takes the characteristics of thermal dissipation rate into consideration in performing the body temperature measurement procedure, in which the measurement method includes selecting a first measurement position surface a1 (e.g., forehead) and a second measurement position surface a2 (e.g., cheek) with different thermal dissipation rates of the subject body, using a contactless temperature measurement device to respectively measure a first sampling temperature t1 of a first measurement position surface a1 and a second sampling temperature t2 of a second measurement position surface a2, computing a difference between the first sampling temperature t1 and the second sampling temperature t2, inputting the difference into a temperature difference compensation function, and computing a temperature difference compensation value. The temperature difference compensation value can be used to correct an error of an original measured temperature value that is generated due to ambient temperature when using the contactless temperature measurement device to measure the facial temperature or the forehead temperature of the subject, such that the obtained measurement temperature can be close to the actual core temperature of the subject.

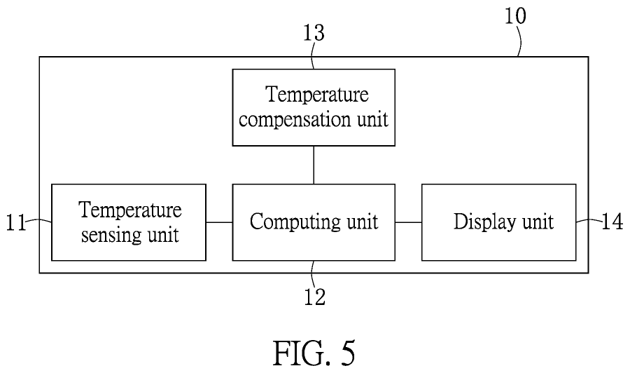

Referring to FIG. 2, which is to be read in conjunction with FIG. 5 to FIG. 10, the body temperature measurement method of the present disclosure includes: a temperature measurement step S1, an original temperature measurement computation step S2, a compensated temperature computation step S3, and a temperature correction step S4.

The temperature measurement step S1 refers to capturing a first sampling temperature t1 of a first measurement position surface a1 of a subject and capturing a second sampling temperature t2 of a second measurement position surface a2 of the subject by a temperature measurement device 10. The first measurement position surface a1 and the second measurement position surface a2 correspond to body surfaces of the subject having different thermal dissipation rates in the air.

In more detail, in the temperature measurement step S1 of this embodiment, the first measurement position surface a1 may be the forehead position of the subject, and the second measurement position surface a2 may be the cheek position of the subject, but the present disclosure is not limited thereto. For example, the first measurement position surface a1 may be the temple position of the side of the face of the subject, while the second measurement position surface a2 may be other positions on the face of the subject, such as the nose or the ears, and other facial position surfaces.

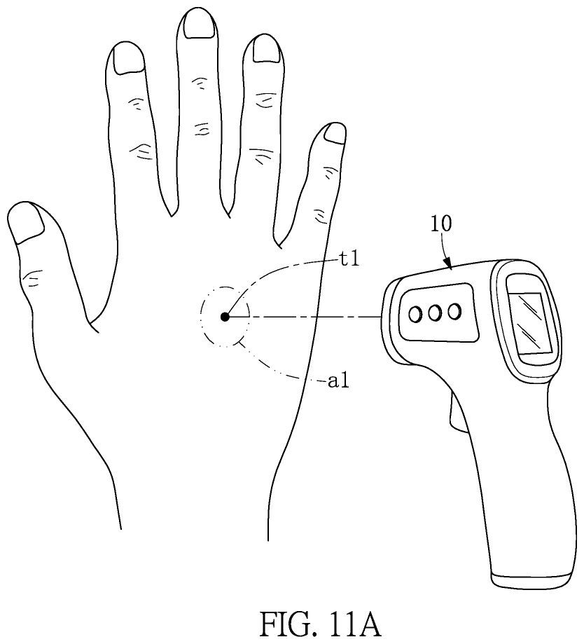

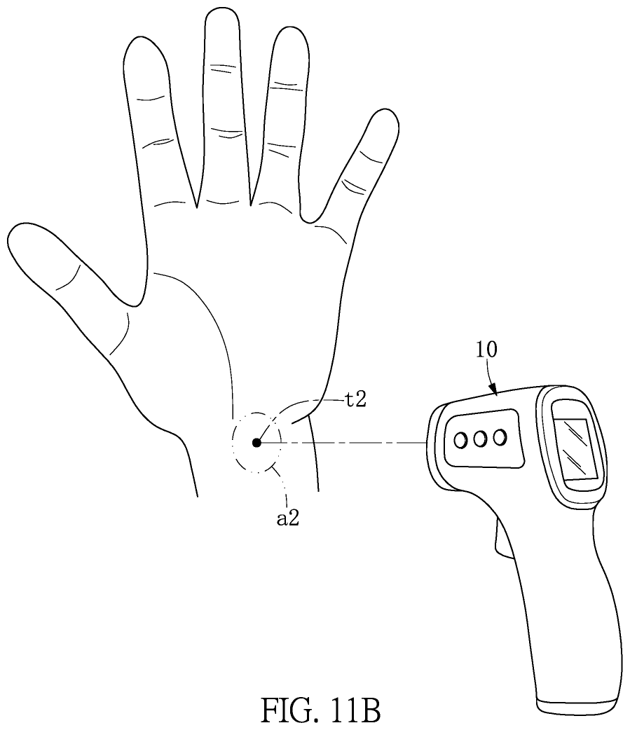

In addition, the first measurement position surface a1 and the second measurement position surface a2 may be located other than on the face of the body, taking the embodiment of FIG. 11A and FIG. 11B as an example, the first measurement position surface a1 may be the back of the hand of the subject, and the second measurement position a2 may be the position of the palm or the wrist of the subject. In the embodiment of FIG. 11A and FIG. 11B, in the temperature measurement step S1, the first measurement position a1 and second measurement position surface a2 are located at the back of the hand and the wrist, in which the first measurement position surface a1 and second measurement position surface a2 have different thermal dissipation rates when the skin contacts the air. Since the first measurement position surface a1 and the second measurement position surface a2 are not located on the head and face of the subject, the embodiment of FIG. 11A and FIG. 11B is particularly suitable for body temperature measurement when the subject is wearing a mask or a hat.

The temperature measurement device 10 used in the step S1 may be an infrared thermometer or an infrared thermal imager. When the infrared thermometer is used to perform the temperature measurement step S1, the infrared thermometer can respectively measure the surface temperatures at a measurement point within the first measurement position surface a1 and at a measurement point within the second measurement position surface a2 (in particular the face) of the subject, so as to obtain the first sampling temperature t1 and the second sampling temperature t2. The infrared thermometer is used to perform the temperature measurement step S1, the infrared thermometer can respectively measure the surface temperatures at a plurality of measurement points within the first measurement position surface a1 and a plurality of measurement points within the second measurement position surface a2, such that a plurality of surface temperatures corresponding to the first measurement position surface a1 can be averaged or processed according to a certain algorithm to obtain the first sampling temperature t1, and a plurality of surface temperatures corresponding to the second measurement position surface a2 can be averaged or processed according to a certain algorithm to obtain the second sampling temperature t2.

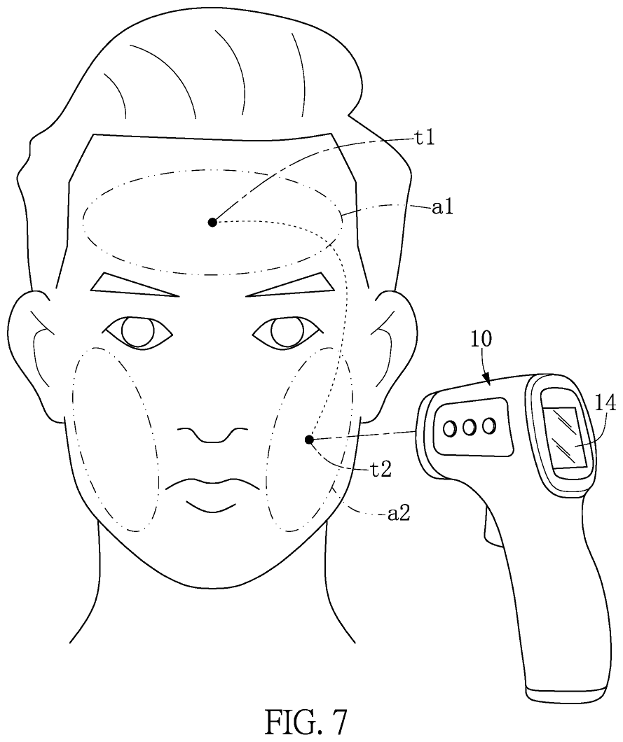

In addition, as shown in FIG. 7, FIG. 7 illustrates a schematic diagram of a manual operation of the temperature measurement device according to the first embodiment of the present disclosure. When the infrared thermal imager is used to perform the temperature measurement step S1, the infrared thermal imager can capture thermal images including the first measurement position surface a1 and the second measurement position surface a2 of the face of the subject by an imaging sensor or a focal plane infrared sensor of an infrared camera device, so that the first sampling temperature t1 and the second sampling temperature t2 can be computed according to the thermal images corresponding to the first measurement position surface a1 and the second measurement position surface a2.

In the original temperature measurement computation step S2, an original measured temperature can be computed according to the first sampling temperature t1 by a computing unit. In the original temperature measurement computation step S2, the computing unit may be a processor integrated in the temperature measurement device 10 or a computer that is separate from the temperature measurement device 10.

The original measured temperature may be regarded as the surface temperature of the first measurement position surface a1 of the subject and the measured value without correction or compensation by the temperature measurement device 10. In this embodiment, the first measurement position can be the forehead of the subject, and thus the original measured temperature is the surface temperature of the forehead position of the subject. Since the temperature measurement device 10 measures the facial surface temperature of the subject, a certain difference exists between the body surface temperature and the actual core body temperature of the subject. In the compensated temperature computation step S3, the computing unit may compute a compensated temperature to correct the original measured temperature, so as to obtain a measured temperature value that is close to the core body temperature of the subject.

In the compensated temperature computation step S3, the computing unit may compute a compensated temperature for correcting the original measured temperature. The compensated temperature computation step S3 includes at least a step of computing a temperature difference compensation value, in which the temperature difference compensation value is computed according to the first sampling temperature t1, the second sampling temperature t2, and a measurement difference. Specifically, the step of computing the temperature difference compensation value includes subtracting the second sampling temperature t2 from the first sampling temperature t1 to obtain a difference value between the first sampling temperature t1 and the second sampling temperature t2, and then inputting the difference between the first sampling temperature t1 and the second sampling temperature t2 into a temperature difference compensation function to obtain the temperature difference compensation value.

The temperature difference compensation function may be obtained by clinical experiments. Specifically, the temperature difference compensation function may be obtained by clinical experiments including measuring, by a contactless temperature measurement device (e.g., infrared thermometer), a plurality of first sampling temperatures t1 at the first measurement position surface a1 and a plurality of second sampling temperatures t2 at the second measurement position surface a2 of a plurality of clinical experimental subjects under different ambient temperatures, respectively, measuring a plurality of core temperatures (e.g., oral temperature) of the plurality of clinical experimental subjects without being influenced by the ambient temperature, such that the plurality of first sampling temperatures t1, the plurality of second sampling temperatures t2, and the plurality of core temperatures of the plurality of clinical experimental subjects can be collected under different ambient temperatures, and then inferring the temperature difference compensation function by regression analysis, computer simulation, or other data analysis methods according to data collected from the clinical experiments.

For example, the following Table 1 includes a plurality of first sampling temperatures t1 at the forehead position, a plurality of second measured temperatures t2 at the cheek position, a plurality of oral measurement temperatures t3 of a plurality of subjects under different ambient temperatures. A first temperature difference Δt1 is obtained by subtracting the second sampling temperature t2 from the first sampling temperature t1, and a temperature measurement sample corresponding to a second temperature difference Δt2 is obtained by subtracting the first sampling temperature t1 from the oral measurement temperature t3.

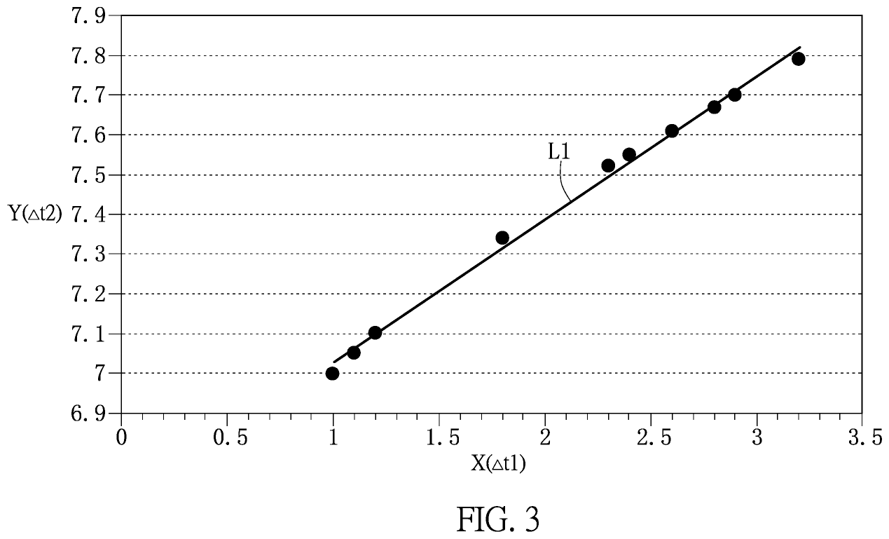

As shown in FIG. 3, FIG. 3 illustrates a two dimensional (X axis and Y axis) coordinate distribution corresponding to the plurality of first temperature differences Δt1 and the plurality of second temperature differences Δt2 in Table 1, the plurality of first temperature differences Δt1 and the plurality of second temperature differences Δt2 are substantially linearly distributed, and a trend curve L1 corresponding to a formula of Y=0.3602X+6.666 can be inferred by linear regression analysis according to the coordinates corresponding to the plurality of first temperature differences Δt1 and the plurality of second temperature differences Δt2. Therefore, the formula corresponding to the linear regression trend curve L1 can be used as the temperature difference compensation function, which can be rewritten into the temperature difference compensation function f(Δt1)=0.3062Δt1+6.666.

TABLE 1
Sampling values of facial temperature and oral temperature
measurement

| | | | | oral | second |
|---|---|---|---|---|---|
| | first | second | first | measure- | temper- |
| | measured | measured | temperature | ment | ature |
| | temper- | temper- | difference | temper- | difference |
| measured | ature | ature | Δt1 (° C) = | ature | Δt2 (° C.) = |
| sample | t1 (° C.) | t2 (° C.) | t1-t2 | t3 (° C.) | Δt3-t1 |
| 1 | 29.4 | 28.3 | 1.1 | 36.45 | 7.05 |
| 2 | 29.1 | 27.9 | 1.2 | 36.2 | 7.1 |
| 3 | 29.6 | 28.6 | 1 | 36.6 | 7 |
| 4 | 29.7 | 28.5 | 1.2 | 36.8 | 7.1 |
| 5 | 29.6 | 28.4 | 1.2 | 36.7 | 7.1 |
| 6 | 27.5 | 24.3 | 3.2 | 35.29 | 7.79 |
| 7 | 27.9 | 25.5 | 2.4 | 35.45 | 7.55 |
| 8 | 28.7 | 25.8 | 2.9 | 36.4 | 7.7 |
| 9 | 28.2 | 25.6 | 2.6 | 35.81 | 7.61 |
| 10 | 28.8 | 26 | 2.8 | 36.47 | 7.67 |
| 11 | 29 | 26.7 | 2.3 | 36.52 | 7.52 |
| 12 | 28.8 | 27 | 1.8 | 36.14 | 7.34 |

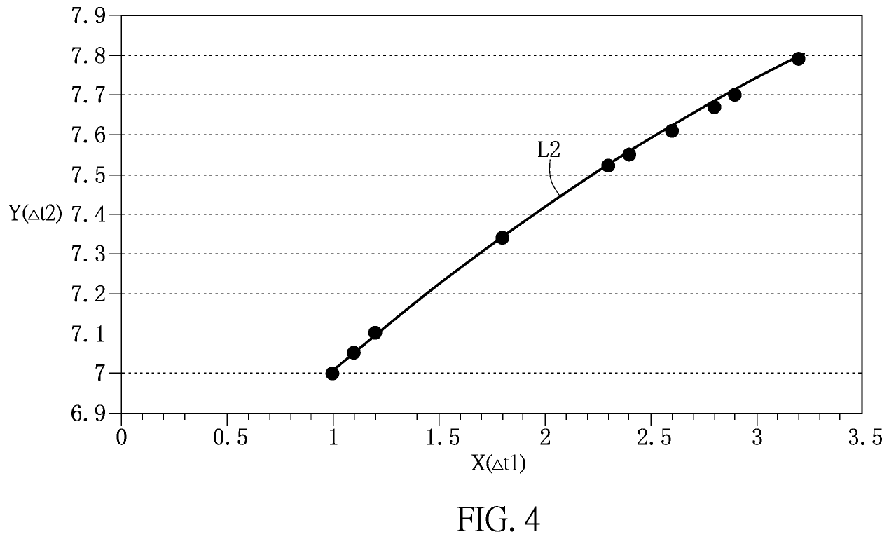

However, it should be noted that the embodiment of the present disclosure can be provided without limiting how the temperature difference compensation value is computed. For example, the temperature difference compensation function may be obtained by polynomial regression analysis, any kind of feasible analysis models, or computer simulation. As shown in FIG. 4, FIG. 4 illustrates a coordinate distribution of experimental data and a trend curve that is obtained according to multiple regression analyses in order to compute a temperature difference compensation function according to an embodiment of the present disclosure. In FIG. 4, a regression curve L2 is obtained according to multiple regression analyses that corresponds to a formula of y=−0.0433x²+0.5346x+6.5169, which can be rewritten into the temperature difference compensation function f(Δt1)=−0.0433Δt1²+0.5346Δt1+6.5169.

In the compensated temperature computation step S3, the compensated temperature further includes an equipment compensation value for correcting an equipment error of the temperature measurement device 10 caused by optical structure, sensor, and other equipment hardware structures. The equipment compensation value may be a constant value, and can be obtained by clinical experiments. In detail, the root cause of the equipment error of the temperature measurement device lies in that the temperature measurement device used for computing the temperature difference compensation function is different from the temperature measurement device 10 used in the temperature measurement method of the present disclosure during the clinical experiments. Therefore, a certain amount of bias must exist between the measured values that are measured by the two temperature measurement devices caused by the optical structure, sensor, and other equipment hardware structures. Accordingly, the present disclosure computes the temperature difference between the measured temperatures from the temperature measurement device 10 of the present disclosure and the measured temperatures from the temperature measurement device of the clinical experiments, so as to obtain the equipment compensation value, such that the equipment error of the temperature measurement device 10 can be corrected according to the equipment compensation value.

In the temperature correction step S4, a measured temperature is obtained according to the original measured temperature obtained by the original temperature measurement computation step S2 and the compensated temperature obtained by the compensated temperature computation step S3, and the measured temperature can be displayed by a display device, so that an examiner can judge whether the body temperature of the subject is normal or not. Therefore, the temperature correction step S4 for computing the outcome of the measured temperature can be represented by the following formula:

T=t0+f(Δt1)+C,

wherein T refers to the measured temperature; t0 refers to the original measured temperature; f(Δt1) refers to the temperature difference compensation value that is computed by inputting the difference Δt1 between the first sampling temperature t1 and the second sampling temperature t2 into the temperature difference compensation function; and C refers to the equipment compensation value for correcting the temperature measurement bias.

In the body temperature measurement method of the present disclosure, the body surface temperatures at different positions correspond to different thermal dissipation rates of the subject that are measured, and the measured temperatures are corrected according to the different thermal dissipation rates corresponding to the different positions, such that the measured temperature is free from the influence of the ambient temperature and the measured temperature can be close to the actual core body temperature.

FIG. 5 illustrates a functional block diagram of a temperature measurement device according to an embodiment of the present disclosure. The temperature measurement device 10 of the present disclosure includes a temperature sensing unit 11, a computing unit 12, a temperature compensation unit 13, and a display unit 14. The temperature sensing unit 11 may be an infrared temperature sensor or thermal imaging equipment, and is configured to measure a first sampling temperature t1 of a first measurement position surface a1 and a second sampling temperature t2 of a second measurement position surface a2 of a subject body by a contactless temperature measurement method. The computing unit 12 is connected to the temperature sensing unit 11, and is configured to compute an original measured temperature according to the first sampling temperature t1 and the second sampling temperature t2 measured by the temperature sensing unit 11. The computing unit 12 further includes a temperature compensation unit 13 built-in with a temperature difference compensation function, and configured to compute a temperature difference between the first sampling temperature t1 and the second sampling temperature and compute a temperature difference compensation value according to the temperature difference compensation function. The computing unit 12 is further configured to obtain a measured temperature according to the temperature difference compensation value for correcting the original measured temperature. The display unit 14 is configured to display the measured temperature, so that an examiner can judge whether the body temperature of the subject is normal or not.

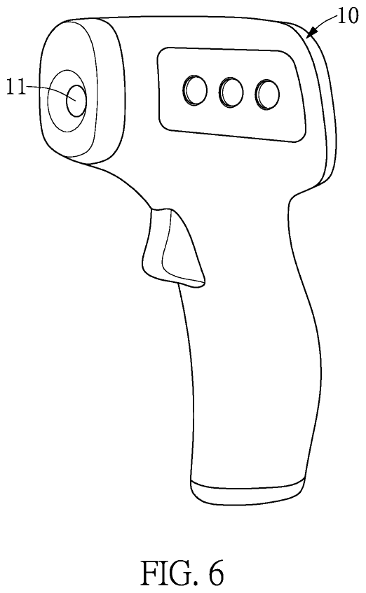

FIG. 6 illustrates a perspective view of the temperature measurement device 10 according to a first embodiment of the present disclosure. In this embodiment, the temperature measurement device 10 may be a handheld infrared thermometer, and the temperature sensing unit 11 disposed in the temperature measurement device 10 of this embodiment includes a sole infrared temperature sensor. As shown in FIG. 7, a user may hold and move the temperature measurement device 10 in the first embodiment to respectively aim the infrared temperature sensor of the temperature sensing unit 11 at the first measurement position surface a1 and the second measurement position surface a2 of the subject body, in order to capture the first sampling temperature t1 and the second sampling temperature t2. Then, after the measured temperature is obtained by the computation of the computing unit and temperature compensation unit, the measured temperature is displayed on the display unit 14.

In particular, in this embodiment, during the operation of the temperature sensing unit 11 of the temperature measurement device 10 measuring the first sampling temperature t1 and the second sampling temperature t2, the measurement method of the present disclosure can be operated in a single-point measurement mode or a multi-point measurement mode. For example, when operating in the single-point measurement mode, the temperature sensing unit 11 of the temperature measurement device 10 respectively measures a single point position within the first measurement position surface a1 and a single point position within the second measurement position surface a2 to capture the first sampling temperature t1 and second sampling temperature t2. When operating in the multi-point measurement mode, as shown in FIG. 7, the user may hold and move the temperature measurement device 10 aiming at the subject body (in particular the face) from the first measurement position surface a1 along an arc shaped trajectory to the second measurement position surface a2, during the movement, the temperature sensing unit 11 captures a plurality of temperatures corresponding to a plurality of measurement points from the first measurement position surface a1 to the second measurement position surface a2, and computes the first sampling temperature t1 and the second sampling temperature t2 by an average method or any other feasible algorithms.

### Second Embodiment

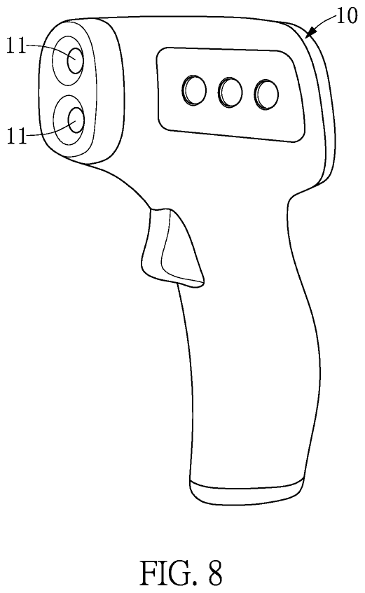

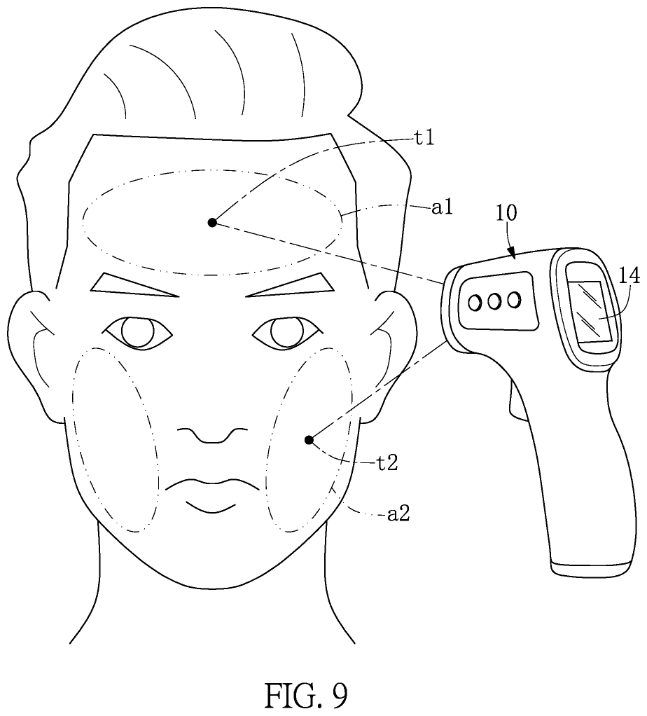

As shown in FIG. 8 and FIG. 9, FIG. 8 illustrates a perspective view of a temperature measurement device according to a second embodiment of the present disclosure, and FIG. 9 illustrates a schematic diagram of a manual operation of the temperature measurement device according to the second embodiment of the present disclosure. In this embodiment, the temperature sensing unit 11 of the temperature measurement device 10 includes at least two infrared temperature sensors, and the two infrared temperature sensors of the temperature sensing unit 11 are able to respectively aim at surfaces of the first measurement position surface a1 and the second measurement position surface a2 of the subject body, such that the two infrared temperature sensor of the temperature sensing unit 11 are able to simultaneously capture the first sampling temperature t1 of the surface of the first measurement position surface a1 and the second sampling temperature t2 of the surface of the second measurement position surface a2 during the temperature measurement procedure.

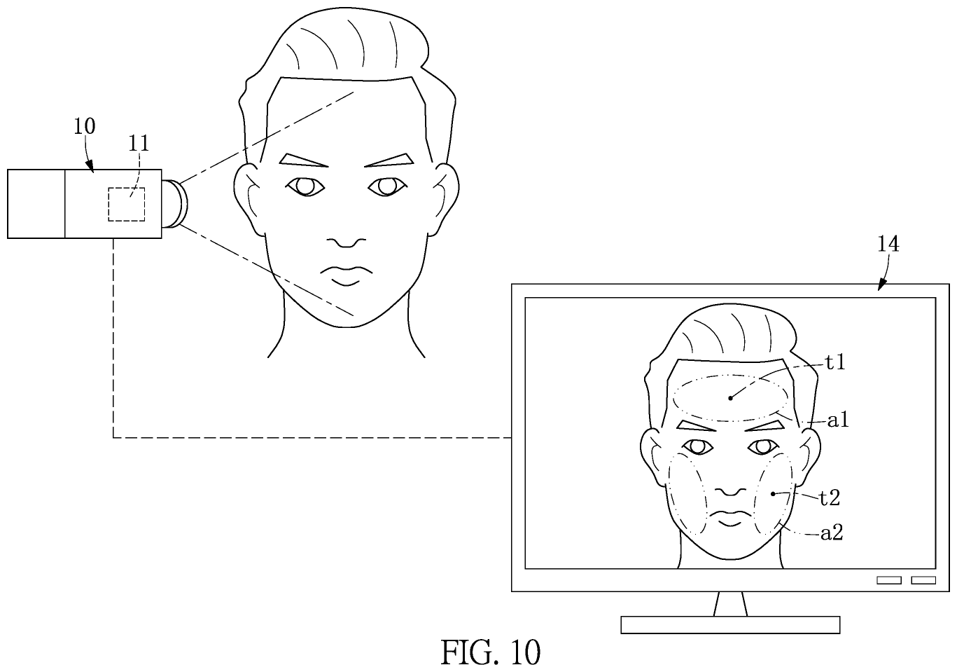

FIG. 10 illustrates another schematic diagram of the manual operation of the temperature measurement device according to the second embodiment of the present disclosure. In this embodiment, the temperature measurement device 10 may be an infrared thermal imager, and the temperature sensing unit 11 may be a thermal image sensor or a focal plane infrared sensor. When using the temperature measurement device 10 in this embodiment, the temperature sensing unit 11 captures the thermal image (in particular the thermal image of the face) of the subject body, the first measurement position surface a1 and the second measurement position surface a2 in the facial thermal image may be recognized by image recognition technology, and the first sampling temperature t1 and the second sampling temperature t2 may be obtained by analyzing the thermal image corresponding to the first measurement position surface a1 and the second measurement position surface a2.

### Advantageous Effects of the Embodiment

An advantageous effect of the present disclosure is that, the temperature measurement method and measurement device of the present disclosure are able to compute temperature difference compensation value according to the temperature difference between different temperatures measured at different positions of the face of the subject, and by correcting the temperature difference compensation value or compensating the original measured temperature, so as to reduce the influence from ambient temperature to the measured temperature, such that the obtained measurement temperature can be closer to the actual core temperature of the subject.

Therefore, when the temperature measurement method and measurement device of the present disclosure is used to perform body temperature measurement, the measurement error generated from the subject being influenced by the ambient temperature can be corrected and the contactless temperature measurement method can be conducted without waiting for the temperature of the subject to return to the temperature that is set by the hypothalamus, so that the convenience and the accuracy of the body temperature measurement operation can be improved.

The foregoing description of the exemplary embodiments of the disclosure has been presented only for the purposes of illustration and description and is not intended to be exhaustive or to limit the disclosure to the precise forms disclosed. Many modifications and variations are possible in light of the above teaching.

The embodiments were chosen and described in order to explain the principles of the disclosure and their practical application so as to enable others skilled in the art to utilize the disclosure and various embodiments and with various modifications as are suited to the particular use contemplated. Alternative embodiments will become apparent to those skilled in the art to which the present disclosure pertains without departing from its spirit and scope.

What is claimed is:

1. A temperature measurement method for measuring a body temperature of a subject to obtain a measured temperature, the temperature measurement method comprising:
capturing, by a temperature measurement device, a first sampling temperature of a first measurement position surface of a subject body and a second sampling temperature of a second measurement position surface of the subject body, the first measurement position surface and the second measurement position surface being where body surfaces of the subject have different thermal dissipation rates when in contact with the air;
computing an original measured temperature according to the first sampling temperature;
computing a compensated temperature, wherein the compensated temperature includes a temperature difference compensation value that is computed according to a difference between the first sampling temperature and the second sampling temperature; and
correcting the original measured temperature according to the compensated temperature to obtain the measured temperature.

2. The method according to claim 1, wherein the first measurement position is located at a forehead position of the subject and the second measurement position is located at a cheek position of the subject.

3. The method according to claim 1, wherein the compensated temperature further includes an equipment compensation value, and the equipment compensation value being a constant value for correcting a fixed equipment error of the temperature measurement device.

4. The method according to claim 1, wherein the temperature measurement device is an infrared thermometer, the infrared thermometer having at least one infrared temperature sensing unit, and the first sampling temperature of the first measurement position surface of the subject and the second sampling temperature of the second measurement position surface of the subject being obtained by the at least one infrared sensing unit.

5. The method according to claim 1, wherein the temperature measurement device is an infrared thermal imager, the step of capturing the first sampling temperature of the first measurement position surface of the subject body and the second sampling temperature of the second measurement position surface of the subject body including:
capturing, by the infrared thermal imager, a thermal image including the subject; and
obtaining the first sampling temperature and the second sampling temperature according to the thermal image corresponding to the first measurement position and the second measurement position of the subject body, respectively.

6. The method according to claim 1, wherein a process of computing the temperature difference compensation value includes inputting the difference between the first sampling temperature and the second sampling temperature into a temperature difference compensation function to compute the temperature difference compensation value.

7. The method according to claim 6, wherein the temperature difference compensation function is inferred according to data including a plurality of differences between a plurality of first sampling temperatures and a plurality of second sampling temperatures captured respectively from the first measurement position surface and the second measurement position surface, and a plurality of actual core temperatures, the plurality of first sampling temperatures and the plurality of second sampling temperatures of a plurality of clinical experimental subjects being measured by a contactless temperature measurement device under a plurality of ambient temperatures.

8. A temperature measurement device configured to measure a body temperature of a subject to obtain a measured temperature, the temperature measurement device comprising:
at least one temperature sensing unit, the at least one temperature sensing unit being an infrared temperature sensor or a thermal image sensor that is configured to capture a first sampling temperature of a first measurement position surface of a subject body, a second sampling temperature of a second measurement position surface of the subject body, and the first measurement position and the second measurement position being positions where body surfaces of the subject have different thermal dissipation rates;
a computing unit connected to the at least one temperature sensing unit and configured to compute an original measured temperature according to the first sampling temperature; and
a temperature compensation unit configured to compute a compensated temperature for correcting the original measured temperature to obtain the measured temperature, the temperature compensation value including a temperature difference compensation value, and the temperature difference compensation value being computed by a temperature difference compensation function according to a difference between the first sampling temperature and the second sampling temperature.

9. The temperature measurement device of claim 8, wherein the compensated temperature of the temperature compensation unit further includes an equipment compensation value, the equipment compensation value being a constant value for correcting a fixed equipment error of the temperature measurement device.

10. The temperature measurement device of claim 8, wherein the temperature measurement device is a handheld infrared thermometer, the temperature sensing unit having at least one infrared temperature sensor, and the first sampling temperature and the second sampling temperature being captured by the temperature sensor that is respectively moved to the first measurement position surface and the second measurement position surface of the subject body.

11. The temperature measurement device of claim 8, wherein the temperature measurement device is a handheld infrared thermometer, the temperature sensing unit having at least two infrared temperature sensors, and the first sampling temperature and the second sampling temperature being captured by two temperature sensors that are respectively aimed at the first measurement position surface and the second measurement position surface of the subject body.

12. The temperature measurement device of claim 8, wherein the temperature measurement device is an infrared thermal imager, the temperature sensing unit being a thermal image sensor or a focal plane infrared sensor configured to capture a thermal image including the subject, and the first sampling temperature and the second sampling temperature are obtained by analyzing the thermal image corresponding to surfaces of the first measurement position surface and the second measurement position surface of the subject body.

13. The temperature measurement device of claim 8, wherein the temperature compensation unit is configured to compute the temperature difference compensation value by inputting the difference between the first sampling temperature and the second sampling temperature into the temperature difference compensation function.

14. The temperature measurement device of claim 13, wherein the temperature difference compensation function is inferred according to data including a plurality of differences between a plurality of first sampling temperatures and a plurality of second sampling temperatures and a plurality of actual core temperatures, the plurality of first sampling temperatures and the plurality of second sampling temperatures of a plurality of clinical experimental subjects being measured by a contactless temperature measurement device under a plurality of ambient temperatures.
